(19) United States
(12) Patent Application Publication
(10) Pub. No.: US 2015/0238466 A1
(43) Pub. Date: Aug. 27, 2015

(54) LIM KINASE INHIBITORS

(71) Applicant: RAMOT AT TEL-AVIV UNIVERSITY LTD., Tel Aviv (IL)

(72) Inventors: Yoel Kloog, Herzliya (IL); Haim Wolfson, Tel Aviv (IL); Shmuel Carmeli, Yehud-Monosson (IL); Efrat Farkash, Givataim (IL); Roni Rak, Ramat Hasharon (IL); Galit Elad-Sfadia, Hod Hasharon (IL); Roni Haklai, Ramat Gan (IL)

(21) Appl. No.: 14/411,141

(22) PCT Filed: Jun. 27, 2013
(86) PCT No.: PCT/IL2013/050555

§ 371 Date: Dec. 24, 2014

Related U.S. Application Data
(60) Provisional application No. 61/665,634, filed on Jun. 28, 2012.

(51) Int. Cl. A61K 31/422; A61K 31/167; A61K 45/06; A61K 31/421

(52) U.S. Cl. CPC A61K 31/422; A61K 31/421; A61K 31/167; A61K 45/06

(57) ABSTRACT

The invention relates to compounds for reducing or inhibiting a biological function mediated by LIMK1 or LIMK2, wherein the compounds are selected to bind the ATP-binding site and/or the substrate-binding site of LIMK.

### TECHNOLOGICAL FIELD

This invention generally relates to LIM kinase inhibitors.

### BACKGROUND

Cell motility is an essential cellular process for embryonic development, wound healing, immune responses and development of tissues. One of the key participants in cell migration is actin, a globular protein which polymerizes into filaments that constitute the basis for cell motion[1].

The actin-depolymerizing factor (ADF)/cofilin family of proteins plays a prominent role in promoting actin depolymerization. At its active, unphosphorylated state, cofilin induces severing (depolymerization) of actin filaments and participates in numerous cellular functions, such as cell migration, cell cycle processes, and neuronal differentiation. Cofilin is phosphorylated mainly by LIM domain kinase 1 (LIMK1) and by LIM domain kinase 2 (LIMK2). At its phosphorylated state, cofilin is inactive and does not affect the cell cytoskeleton. Hyperphosphorylation of cofilin typically occurs in many human diseases and pathological conditions, such as cancer cell invasion and metastasis, as well as in neurodevelopmental disorders, for example Williams syndrome.

Ras inhibition by the Ras inhibitor S-trans, trans-Farnesyl Thio Salicyclic acid (FTS; Salirasib) in neurofibromin (NF1^{-/-}) cells inhibits their motility and spreading, alters gene expression, and eliminates the expression of regulators of cell-matrix interaction[1].

The first LIMK inhibitor to be discovered was N-{5-(2-(2,6-dichloro-phenyl)-5-difluoromethyl-2H-pyrazol-3-yl)-thiazol-2-yl}-isobutyramide (compound 3 in [2], hereafter referred to as BMS-5); BMS-5 inhibits both LIMK1 and LIMK 2 [3].

International applications published under WO 2009/021169 and WO 2009/131940 [4] generally pertain to pyrrole-pyrimidine-based inhibitors of LIM kinase 2, compositions comprising them and methods of their use.

### REFERENCES

[1] Barkan, B., et al., Clin. Cancer Res. 12:5533-5542 (2006)
[2] Ross-Macdonald, P., et al., Mol. Cancer Ther. 7:3490-3498 (2008)
[3] WO 2009/021169
[4] WO 2009/131940
[5] Roy, A., et al., Nat. Protoc. 5:725-738 (2010)
[6] Yoshioka, K., et al., PNAS 100(12):7247-7252 (2003)
[7] Starinsky-Elbaz, S., et al., Mol. Cell Neurosci. 42: 278-287 (2009)
[8] Wallace, M. R., et al., Science 249:181-186 (1990)
[9] Marchuk, D. A., et al., Genomics 11:931-940 (1991)
[10] Buchberg, A. M., et al., Nature 347:291-294 (1990)
[11] Cichowski, K. and Jacks, T. Cell 104:593-604 (2001)
[12] Altschul, S. F., et al., J. Mol. Biol. 215:403-10 (1990)
[13] Irwin, J. J. and Shoichet, B. K., J. Chem. Inf. Model. 45:177-182 (2005)
[14] Shapira, S., et al., Cell Death Differ. 14(5):895-906 (2007)
[15] Zhao, L., et al., Front Biosci. (Elite Ed) 2: 241-249 (2010)
[16] Raftopoulou, M., and Hall, A. Dev Biol 265:23-32 (2004)
[17] Etienne-Manneville, S. Methods Enzymol. 406:565-578 (2006)
[18] Goldberg, L. and Kloog, Y., Cancer Res. 66:11709-11717 (2006)
[19] Eswar, N. et al., Curr. Protoc. Protein. Sci. Chapter 2:Unit 29 (2007)

### SUMMARY OF THE INVENTION

Cancer cells may acquire the ability to penetrate and infiltrate surrounding normal tissues, i.e., to migrate or metastasize, forming a new tumor. Thus, inhibiting or reducing the ability of cancer cells to migrate is of a highly therapeutic value.

The inventors of the present invention have found that compounds which bind to at least one of (a) the substrate binding site and (b) the ATP binding site of LIM kinase (LIMK) are effective in the treatment of disease states mediated by LIMK. Thus, effective compounds in accordance with the invention are those capable of binding both to amino acids constituting the substrate binding site and the ATP binding site of LIMK1, e.g., as depicted in PDB ID: 3S95, and/or the predicted substrate binding site and ATP binding site of LIMK2.

Thus, in one aspect of the invention, there is provided a compound for reducing or inhibiting a biological function mediated by LIMK1 or LIMK2, said compound being selected to bind the ATP-binding site and/or the substrate-binding site of LIMK.

In some embodiments, the compound capable of binding to the ATP-binding site and the substrate-binding site of LIMK is a compound of Formula (I). As further disclosed herein, the reduction or inhibition of the LIMK biological function was demonstrated by reduction in the phosphorylation of cofilin, accompanied by actin severing and inhibition of cell migration, reduction in cell proliferation, and reduction in anchorage-independent colony formation in soft agar of NF1^{-/-} MEFs cells.

The inventors have also demonstrated that compounds of the general Formula (I) are effective in reducing or inhibiting LIMK biological function in general. Thus, in another aspect, the present invention contemplates a compound of Formula (I), and pharmaceutically acceptable salt(s) thereof:

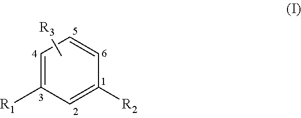

wherein

R₁ and R₂, each independently of the other, is selected from —NHC(═O)R₄ and —C(═O)NHR₅;

R₃ (being position at any one or more of the ring carbon atoms, may be 1, 2, 3 or 4 same or different groups) is selected from —H and C₁-C₆alkyl;

R₄ and R₅, each independently of the other, is selected from a substituted or unsubstituted C₆-C₁₂aryl, substituted or unsubstituted C₃-C₅heteroaryl and substituted or unsubstituted C₃-C₅heterocyclyl;

the compound of Formula (I) being for use in a method of reducing or inhibiting a biological function mediated by LIM Kinase (LIMK).

As known in the art, LIM kinase (LIMK) is a protein kinase having a LIM protein domain (LIM domain, named after its initial discovery in the proteins Lin11, Isl-1 & Mec-3) composed of two contiguous zinc finger domains, separated by a two-amino acid residue hydrophobic linker. In some embodiments, the LIM kinase is LIM kinase-1 (LIMK1). In other embodiments, the LIM kinase is LIM kinase-2 (LIMK2). Thus, the present invention pertains to reducing or inhibiting a biological function mediated by LIMK1 or LIMK2.

The “biological function mediated by LIMK” refers to any cellular activity, which is mediated or regulated by LIMK. The biological functions mediated by LIMK according to the present invention include the direct activity of LIMK in phosphorylating actin-depolymerizing factor cofilin, which results in cofilin inactivation, leading to increased cell motility, and the indirect involvement of LIMK in multiple cellular activities mediated by cofilin, namely actin cytoskeleton reorganization, cell proliferation, cell migration, cell motility, anchorage-independent cell growth, and tumor progression and metastasis.

As stated above, a compound of Formula (I) is intended for use in reducing or inhibiting a biological function mediated by LIMK. The term “reducing” refers to a complete or partial restriction, retardation, decrease or diminishing of the biological function mediated by LIMK. In some embodiments, said biological function mediated by LIMK is inhibited, namely is completely restricted, suppressed or diminished.

The compounds of Formula (I) share a central benzene ring structure substituted as shown above. In Formula (I):

(a) R₁ and R₂, independently of the other, are selected from —NHC(═O)R₄ and —C(═O)NHR₅. As used herein, the group “—NHC(═O)R₄” refers to an amide group, wherein the nitrogen atom is connected to the benzene ring, and to a hydrogen atom and to a carboxyl (—C(═O)) group, which in turn is connected to variant R₄. Similarly, the group “—C(═O)NHR₅” refer to an amide group, wherein the carboxyl group (—C(═O)) is connected to the benzene ring and to a nitrogen atom, which in turn is connected to a hydrogen atom and to variant R₅. In some embodiments, the nitrogen atom may be further protonated or alkylated by C₁-C₆alkyl to give a quaternary amide.

(b) R₃ is selected from —H (hydrogen atom) and C₁-C₆alkyl. As used “C₁-C₆ alkyl” refers to an alkyl group, having between 1 and 6 carbon atoms, which may be linear or branched. Non-limiting examples of such alkyl group include methyl, ethyl, propyl, iso-propyl, iso-butyl, n-butyl, sec-butyl, tert-butyl, n-pentyl, 2-pentyl, 3-pentyl, n-hexyl, 2-hexyl, 3-hexyl and others. In some embodiments, said C₁-C₆alkyl is methyl, ethyl, propyl, n-butyl, n-pentyl or n-hexyl. In other embodiments, the C₁-C₆alkyl is an aliphatic group containing 1, 2, 3, 4, 5, or 6 carbon atoms.

In further embodiments, said C₁-C₆alkyl is methyl or ethyl.

As shown in the structure of Formula (I), R₃ may be substituted at (bonded to) any one or more of the ring carbons, and R₃ represents one, two, three or four substituting groups. In some embodiments, where R₃ is a single group, it may be substituted at position 2, 4, 5 or 6 (para-, meta- or ortho- to R₁ (or R₂), or at the position between R₁ and R₂), as depicted in the structure of Formula (I) above. Where R₃ represents two groups (designated R₃¹ and R₃²), the two groups may be at positions (2 and 4), (2 and 5), (2 and 6), (4 and 5), (4 and 6), or (5 and 6). Where R₃ represents three groups (designated R₃¹, R₃² and R₃³), the three groups may be substituted at positions (2, 4 and 5), (2, 5 and 6), (4, 5 and 6) on the benzene ring. Where R₃ represents four groups (designated R₃¹, R₃², R₃³ and R₃⁴), all ring positions (2, 4, 5 and 6) are substituted.

In some embodiments, R₃ represents one group. In other embodiments, R₃ represents two groups, R₃¹ and R₃². In some embodiments, each of R₃ is —H. In other embodiments, where R₃ represents two, three or four groups, at least one of the groups is —H.

(c) R₄ and R₅ are selected, independently of each other, from a substituted or unsubstituted C₆-C₁₂aryl, substituted or unsubstituted C₃-C₅heteroaryl and substituted or unsubstituted C₃-C₅heterocyclic.

As used herein, the expression “C₆-C₁₂aryl” refers to an aromatic monocyclic or multicyclic group containing from 6 to 12 carbon atoms. In some embodiments, the aryl group is fluorenyl. In other embodiments, the aryl is phenyl. In further embodiments, the aryl group is naphthyl.

The “C₃-C₅heteroaryl” is a monocyclic or multicyclic aromatic ring system having between 3 and 5 carbon atoms and at least one heteroatom selected from N, O and S in the ring system. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

The “C₃-C₅heterocyclic” is a monocyclic or multicyclic non-aromatic ring system having between 3 and 5 carbon atoms and at least one heteroatom selected from N, O and S in the ring system. In some embodiments, at least one of the heteroatom is nitrogen and/or oxygen.

As stated above, each of the variants C₆-C₁₂aryl, C₃-C₅heteroaryl and C₃-C₅heterocyclic may be substituted or unsubstituted. Where a specific variant is substituted, such substitution may be of an atom or group selected from —H, halide (I, Br, Cl, F), —CF₃, hydroxyl (—OH), amine (—NH₃ or primary, secondary, tertiary or quarternized amine), nitro (—NO₂), C₁-C₆alkyl, C₁-C₆alkoxy (an alkylene or alkyl substituted by —O—), etc.

In some embodiments, each of R₄ and R₅, independently of the other is selected from phenyl, naphthyl, isoxazolyl, and oxazolyl. In further embodiments, each of R₄ and R₅, independently of the other, is isoxazolyl, optionally substituted with a C₁₋₆alkyl. In further embodiments, each of R₄ and R₅, independently of the other, is isoxazolyl substituted with methyl.

In some embodiments, wherein in a compound of Formula (I), R₁ is —NHC(═O)R₄, the compound is a compound of Formula (II):

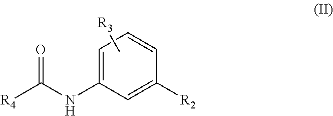

wherein R₂, R₃ and R₄ are as defined above.

In some embodiments, in a compound of Formula (I), R₁ is —C(═O)NHR₅, the compound is a compound of Formula (III):

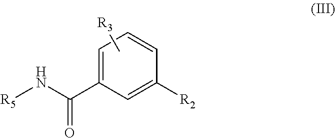

wherein R₂, R₃ and R₅ are as defined above.

In some embodiments, in a compound of Formula (III), R₂ is selected from —NHC(═O)R₄ and —C(═O)NHR₅. In some embodiments, R₂ is —NHC(═O)R₄. In other embodiments, R₂ is —C(═O)NHR₅, the compound being a compound of Formula (IIIa):

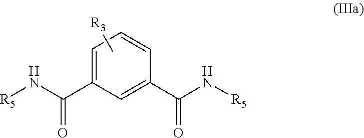

wherein R₂ and R₅ are as defined above.

In some embodiments, in a compound of Formula (IIIa), R₃ is as defined above and each of the two R₅ groups may be same or different.

In some embodiments, one R₅ is a substituted phenyl group, e.g., said substitution being selected from with at least one group selected from —CF₃, C₁-C₆alkyl, and isoxazolyl optionally substituted with C₁₋₆alkyl, and the other R₅ being selected from selected from phenyl, naphthyl, isoxazolyl, and oxazolyl.

In some embodiments, one of said R₅ is isoxazolyl optionally substituted with C₁₋₆alkyl, and the other R₅ is —CF₃ substituted phenyl.

Wherein, in a compound of Formula (II), R₂ is —NHC(═O)R₄, the compound is a compound of Formula IV:

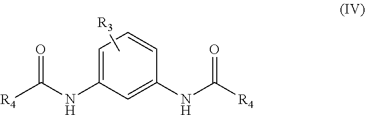

wherein R₃ and R₄ are selected as above and wherein each of R₄ may the same or different (where the two R₄ groups are different, one R₄ group is labeled “R₄” and the other “R₄¹”).

In some embodiments, the two R₄ groups are not the same; thus, the compound of Formula (IV) is a compound of Formula (IVa):

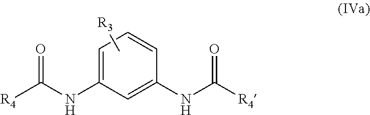

wherein R₃ and R₄ are as defined above and R₄¹ is selected from a substituted or unsubstituted C₆-C₁₂aryl, substituted or unsubstituted C₃-C₅heteroaryl and substituted or unsubstituted C₃-C₅heterocyclic.

In some embodiments, each of R₄ and R₄¹, independently of the other, is selected from substituted or unsubstituted C₆-C₁₂aryl.

In some embodiments, R₄ is a substituted or unsubstituted naphthyl and each of R₃ and R₄¹ are as defined above. Thus, the compound of Formula (IVa) is a compound of Formula (IVb):

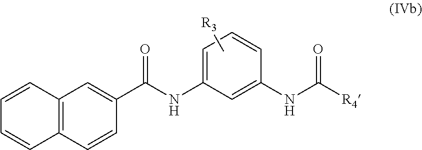

wherein R₃ and R₄¹ are each as defined hereinabove.

In some embodiments, in a compound of Formula (IVa) R₄ is a substituted or unsubstituted phenyl and each of R₃ and R₄¹ are as defined above.

In some embodiments, in a compound of Formula (IVa) each of R₄ and R₄¹, independently of the other, is selected from substituted or unsubstituted C₃-C₅heterocyclic.

In some embodiments, R₄ is isoxazolyl or oxazolyl, or naphthalenyl, each being substituted or unsubstituted. In further embodiments, R₄ is isoxazolyl optionally substituted with C₁₋₆alkyl. Thus, in some embodiments, the compound of Formula (IVa) is a compound of Formula (IVc):

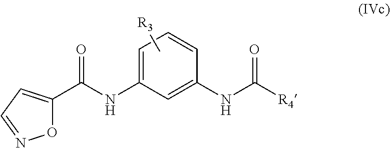

wherein each of R₃ and R₄¹ are as defined above.

In further embodiments, the isoxazolyl (substituted at R₄) is substituted with a C₁₋₆ alkyl, e.g., methyl; the compound thus being a compound of Formula (IVd):

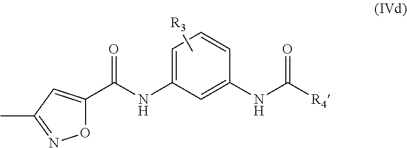

wherein each of R₃ and R₄¹ are as defined above.

In some embodiments, in a compound of Formula (IVb) and/or (IVc) and/or (IVd), R₄¹ is substituted or unsubstituted phenyl. In some embodiments, R₄¹ is a phenyl substituted with at least one group selected from C₁₋₆alkyl, C₁₋₆cycloalkyl, substituted or unsubstituted imidazolidine. In some embodiments, R₄¹ is phenyl substituted with cyclohexane or methyl or ethyl or propyl, or butyl, or imidazolidine, or imidazolidine substituted with methyl.

In some embodiments, in a compound of Formula (IVb) and/or IVc) and/or (IVd), R₄¹ is a substituted phenyl. In some embodiments, R₄¹ is a phenyl substituted with at least one group selected from —CF₃, C₁-C₆alkyl, and substituted or unsubstituted imidazolidine. In some embodiments, R₄¹ is phenyl substituted with cyclohexane or methyl or ethyl or propyl, or butyl, or imidazolidine, or imidazolidine substituted with methyl.

In some embodiments, in a compound of Formula (IVb) and/or IVc) and/or (IVd), R₄¹ is a substituted phenyl, said substitution being selected from —CF₃, methyl, ethyl, cyclohexyl, imidazolidine, and imidazolidine substituted with methyl.

In some embodiments, in a compound of Formula (IVb) and/or IVc) and/or (IVd), R₄¹ is a —CF₃ substituted phenyl.

In some embodiments, in a compound of Formula (IVb) and/or IVc) and/or (IVd), R₄¹ is a methyl substituted phenyl.

In some embodiments, in a compound of Formula (IVb) and/or IVc) and/or (IVd), R₄¹ is a cyclohexyl substituted phenyl.

In some embodiments, in a compound of Formula (IVb) and/or IVc) and/or (IVd), R₄¹ is an imidazolidine substituted phenyl, said imidazolidine being optionally also substituted with methyl.

In some embodiments, in a compound of Formula (IVb) and/or IVc) and/or (IVd), R₄¹ is a substituted phenyl, said substitution being by two groups, each being selected from —CF₃, methyl, ethyl, cyclohexyl, imidazolidine, and imidazolidine substituted with methyl.

In some embodiments, in a compound of Formula (IVb) and/or IVc) and/or (IVd), R₄¹ is a substituted phenyl, said substitution being by —CF₃ and methyl

In some embodiments, in a compound of Formula (IVb) and/or IVc) and/or (IVd), R₄¹ is a substituted phenyl, said substitution being by —CF₃ and cyclohexyl.

In some embodiments, in a compound of Formula (IVb) and/or IVc) and/or (IVd), R₄¹ is a substituted phenyl, said substitution being by —CF₃ and imidazolidine, or —CH₃ and imidazolidine substituted with methyl.

In some embodiments, in a compound of Formula (IVa), R₃ is hydrogen or methyl, R₄ is isoxazolyl substituted with methyl, R₄¹ is phenyl substituted with cyclohexane or with methyl or with imidazolidine (which may be further methylated).

In some embodiments, in a compound of Formula (III), R₂ is —NHC(═O)R₄, wherein R₄ is as defined above. Thus, the compound of Formula (III) is a compound of Formula (V):

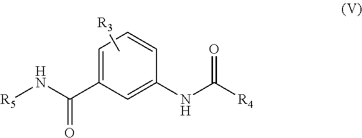

wherein each of R₃, R₄ and R₅ are as defined hereinabove.

In some embodiments, in a compound of Formula (V), R₄ is substituted or unsubstituted phenyl. In further embodiments, R₄ is phenyl substituted with —CF₃. In further embodiments, R₄ is phenyl substituted with CF₃ at any of the phenyl ring positions.

In some embodiments, in a compound of Formula (V), R₅ is substituted or unsubstituted phenyl. In further embodiments, R₅ is phenyl substituted with —CF₃. In further embodiments, R₅ is phenyl substituted with —CF₃ at any of the phenyl ring positions. In some embodiments, the position of substitution of said —CF₃ is meta- to the amide nitrogen.

In some embodiments, the compound of Formula (V), R₄ is substituted or unsubstituted C₃-C₅heteroaryl. In some embodiments, R₄ is substituted C₃-C₅heteroaryl. In further embodiments, R₄ is substituted C₃-C₅heteroaryl substituted with C₁₋₆ alkyl. In further embodiments, R₄ is substituted C₃-C₅heteroaryl substituted with methyl. In further embodiments, R₄ is isoxazole substituted with methyl.

In some embodiments, in a compound of Formula (V), R₅ is phenyl substituted with —CF₃ at any of the phenyl ring positions and R₄ is substituted C₃-C₅heteroaryl substituted with methyl. In further embodiments, R₄ is isoxazole substituted with methyl.

In some embodiments, the compound of Formula (V) is Compound 1, Compound 14, Compound 15, Compound 16 and Compound 17.

In some embodiments, the compound of Formula (III), R₅ is substituted or unsubstituted C₃-C₅heteroaryl. In some embodiments, R₅ is substituted C₃-C₅heteroaryl. In further embodiments, R₅ is substituted C₃—Csheteroaryl substituted with C₁₋₆ alkyl. In further embodiments, R₅ is substituted C₃-C₅heteroaryl substituted with methyl. In further embodiments, R₅ is isoxazole substituted with methyl.

In some embodiments, in all compounds of the above recited formulae, R₃ may be —H or may be a C₁-C₆alkyl. In some embodiments, R₃ is —H or a methyl group.

In some embodiments, in all compounds of the above recited formulae, R₃ represents a single substituent at position 2, 4, 5, or 6. In some embodiments, in all compounds of the above recited formulae, R₃ represents a single substituent at position 2, 4, 5, or 6.

In some embodiments, in all compounds of the above recited formulae, R₃ represents a single substituent at position 2, 4, 5, or 6.

In some embodiments, in all compounds of the above recited formulae, R₃ represents a single substituent at position 2.

In some embodiments, in all compounds of the above recited formulae, R₃ represents a single substituent at position 4.

In some embodiments, in all compounds of the above recited formulae, R₃ represents a single substituent at position 5.

In some embodiments, in all compounds of the above recited formulae, R₃ represents a single substituent at position 6.

In further embodiments, the compounds utilized in accordance with the invention are compounds designated in Table 1 as Compound 1, 2, 3, 4, 5, 6, 7, 8, 9, 10, 11, 12, 13, 14, 15, 16, and 17. In other embodiments, the compounds are Compounds designated in Table 1 as Compound 1 and/or 2 and/or 3 and/or 4 and/or 5 and/or 6 and/or 7 and/or 8 and/or 9 and/or 10 and/or 11 and/or 12 and/or 13 and/or 14 and/or 15 and/or 16 and/or 17. In some embodiments, the compounds of the invention are compounds designated in Table 1 as Compound 1.

TABLE 1
Compounds utilized in accordance with the invention

| Compound no. | Structure |
|---|---|
| 1 (T56-LIMKi) | 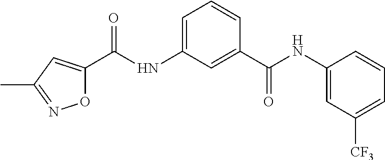 [SMILES: Cc1cc(C(=O)Nc2cccc(C(=O)Nc3cccc(C(F)(F)F)c3)c2)on1] |
| 2 (AWL-II-38.3) | 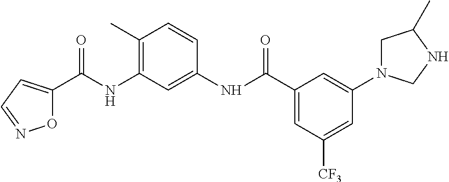 [SMILES: Cc1ccc(NC(=O)c2cc(N3CNC(C)C3)cc(C(F)(F)F)c2)cc1NC(=O)c1ccno1] |
| 3 | 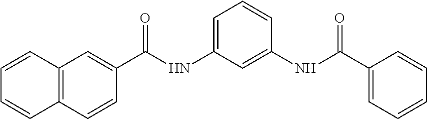 [SMILES: O=C(Nc1cccc(NC(=O)c2ccc3ccccc3c2)c1)c1ccccc1] |
| 4 | 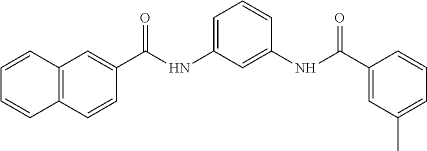 [SMILES: Cc1cccc(C(=O)Nc2cccc(NC(=O)c3ccc4ccccc4c3)c2)c1] |
| 5 | 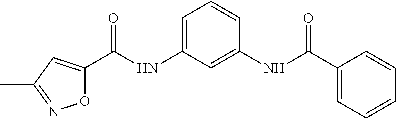 [SMILES: Cc1cc(C(=O)Nc2cccc(NC(=O)c3ccccc3)c2)on1] |
| 6 | 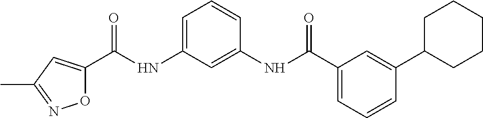 [SMILES: Cc1cc(C(=O)Nc2cccc(NC(=O)c3cccc(C4CCCCC4)c3)c2)on1] |
| 7 | 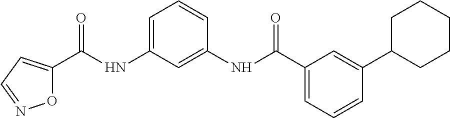 [SMILES: O=C(Nc1cccc(NC(=O)c2ccno2)c1)c1cccc(C2CCCCC2)c1] |
| 8 | 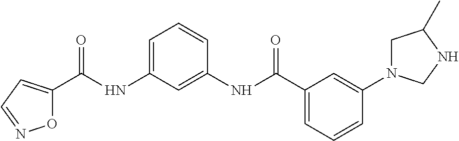 [SMILES: CC1CN(c2cccc(C(=O)Nc3cccc(NC(=O)c4ccno4)c3)c2)CN1] |
| 9 | 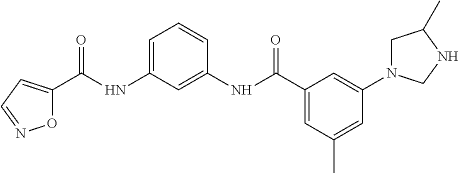 [SMILES: Cc1cc(C(=O)Nc2cccc(NC(=O)c3ccno3)c2)cc(N2CNC(C)C2)c1] |
| 10 | 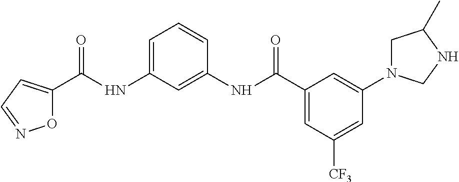 [SMILES: CC1CN(c2cc(C(=O)Nc3cccc(NC(=O)c4ccno4)c3)cc(C(F)(F)F)c2)CN1] |
| 11 | 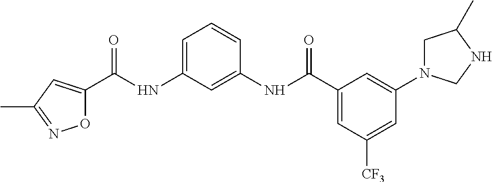 [SMILES: Cc1cc(C(=O)Nc2cccc(NC(=O)c3cc(N4CNC(C)C4)cc(C(F)(F)F)c3)c2)on1] |
| 12 | 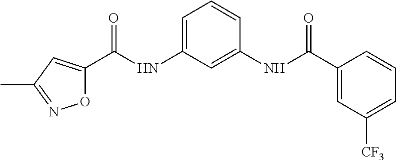 [SMILES: Cc1cc(C(=O)Nc2cccc(NC(=O)c3cccc(C(F)(F)F)c3)c2)on1] |
| 13 | 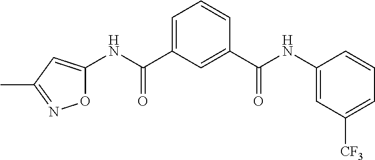 [SMILES: Cc1cc(NC(=O)c2cccc(C(=O)Nc3cccc(C(F)(F)F)c3)c2)on1] |
| 14 | 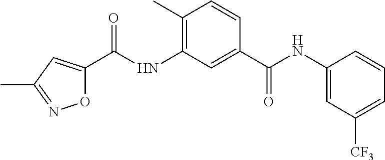 [SMILES: Cc1cc(C(=O)Nc2cc(C(=O)Nc3cccc(C(F)(F)F)c3)ccc2C)on1] |
| 15 | 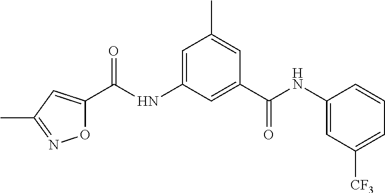 [SMILES: Cc1cc(NC(=O)c2cc(C)no2)cc(C(=O)Nc2cccc(C(F)(F)F)c2)c1] |
| 16 | 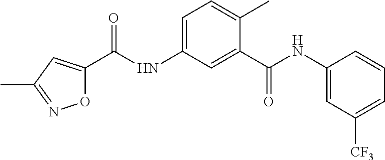 [SMILES: Cc1cc(C(=O)Nc2ccc(C)c(C(=O)Nc3cccc(C(F)(F)F)c3)c2)on1] |
| 17 | 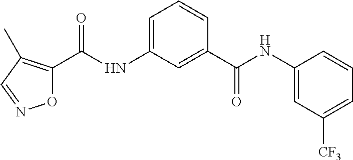 [SMILES: Cc1cnoc1C(=O)Nc1cccc(C(=O)Nc2cccc(C(F)(F)F)c2)c1] |

In some embodiments, the compounds of Formula (I) or Table 1 are utilized in reducing or inhibiting a biological function specifically mediated by LIM Kinase. In some embodiments, the compound is compound herein designated Compound 1.

The compounds utilized in accordance with the present invention may be used in their free base or free acid form or as “pharmaceutically acceptable salt(s)”, namely as salts that are safe and effective for pharmaceutical use in mammals (e.g., humans) and that possess the desired biological activity.

Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. SCI. 1-19 (1977).

In another aspect of the present invention, there is provided the use of at least one compound of any of the above Formulae for the preparation of a pharmaceutical composition for use in reducing or inhibiting a biological function mediated by LIM Kinase (LIMK).

In another aspect, the invention provides a pharmaceutical composition comprising at least one compound of any one of the above Formulae for use in reducing or inhibiting a biological function mediated by LIM Kinase (LIMK). Namely, the compounds used as disclosed herein may reduce or inhibit one or more of the following:
phosphorylation of cofilin—the level of cofilin phosphorylation may be determined by any protocol known to a person skilled in the field of the invention. A specific non limited example for determining the phosphorylation level of a protein is western blot analysis, employing a specific antibody directed against the phosphrylated protein;
cell proliferation—increase in the number of cells as a result of cell growth and cell division. Cell proliferation may be followed by any method known in the field of the invention. Examples for monitoring cell proliferation include, but are not limited to, direct observation or monitoring of the secretion of various cytokines, which are indicative of the cell proliferation state or profile or assessing the variation in cell number in a cell culture (by counting);
cell migration—movement of a tissue, formation during embryonic development, wound healing and immune responses;
cell motility—ability of cells to move spontaneously and actively, consuming energy in the process. The level of cell motility or migration may be determined by, for example, following the level of growing actin fibers, which is a measure of cell motility. The level of growing actin fibers may be monitored by any procedure known to a person skilled in the art, for example, by monitoring the fluorescent labeling of actin (e.g., using fluorescein phalloidin, rhodamine phalloidin, etc.). In some embodiments, the level of growing actin fibers may be monitored by following the fluorescence of actin monomers, labeled with pyrene iodoacetamide, which has been demonstrated to change upon polymerization;
anchorage-independent cell growth—cell population that is capable of proliferating independently of both external and internal signals. Monitoring anchorage-independent cell growth may be performed by any method known to those of skill in the art, for example, by performing soft agar assays; and/or
tumor progression and metastasis—the last phase in tumor development. This phase is characterized by increased growth speed and in the invasiveness of the tumor cells (metastasis). Tumor invasion (or metastasis) may be examined by any method known in the field of the invention. For example, metastasis may be followed in vivo by standard imaging. As a non-limiting example, cell migration may be monitored by a scratch-induced migration assay.

In some embodiments, the pharmaceutical composition according to the invention further comprises an additional therapeutic agent. As used herein, the term “therapeutic agent” refers to any agent that is known, clinically shown, or expected by clinicians to provide a therapeutic benefit for reducing or inhibiting a pathological condition, when provided in a therapeutically effective amount.

In some embodiments, the therapeutic agent is selected from an anti-proliferative agent, a cytotoxic agent, a cytokine, a hormone, and an antibody.

In some embodiments, the therapeutic agent is an anti-proliferative agent, selected to inhibit cancer cell growth. In some embodiments, the anti-proliferative agent is farnesyl thiosalicyclic acid (FTS, Salirasib).

In some embodiments, the therapeutic agent is a cytotoxic agent selected to inhibit or prevent the function of cells and/or cause destruction of cells. In some embodiments, the cytotoxic agent is selected from a radioactive agent, a toxin, an antimetabolite, and an alkylating agent.

In some embodiments, the therapeutic agent is a cytokine. Examples of cytokines encompassed by the invention may be, but are not limited to, immunomodulating agents, such as interleukines and interferons. Also encompassed are lymphokines and chemokines.

In some embodiments, the therapeutic agent is a hormone, which is able to inhibit the growth of tumor cell, or a hormone which is able to induce apoptosis (programmed cell death).

In some other embodiments, the therapeutic agent is an antibody, selected from a polyclonal antibody, a monoclonal antibody, a chimeric antibody, a humanized antibody, a human antibody, or any fragment thereof, which retains the binding activity of the antibody. In some embodiments, the antibody is a neutralizing antibody (i.e. an antibody, which reacts with an antigen, and inhibits or antagonizes its biological activity).

The composition of the invention may additionally comprise at least one inert agent selected from a buffering agent, an agent which adjusts the osmolarity thereof, a pharmaceutically acceptable carrier, excipient and/or diluents.

The pharmaceutically acceptable carriers, vehicles, adjuvants, excipients, or diluents, are well-known to those skilled in the art and are readily available to the public. It is preferred that the pharmaceutically acceptable carrier be one which is chemically inert to the active compounds and one which has no detrimental side effects or toxicity under the conditions of use.

The choice of a carrier will be determined in part by the particular active agent, as well as by the particular method used to administer the composition. The carrier can be a solvent or a dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

In an additional aspect of the invention, there is provided a pharmaceutical composition according to the invention for use in a method of prophylaxis or treatment of a disease state or condition mediated by LIMK.

As used herein, the term “prophylaxis or treatment” refers to the administering of a therapeutic amount of the composition of the present invention which is effective to ameliorate undesired symptoms associated with a disease state or condition mediated by LIMK, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

In some embodiments, the disease state or condition mediated by LIMK is a disease state or condition mediated by LIMK2.

The “disease state or condition mediated by LIMK” refers to any abnormal condition of the body that causes discomfort, dysfunction, or distress to the person affected that is associated with the activity of LIMK. Examples include, but are not limited to, proliferative disorders, disorders associated with neuronal differentiation, e.g. neurodevelopmental disorders (for example Williams syndrome) and neurofibromatosis.

In some embodiments, the disease state or condition mediated by LIMK is a proliferative disease. In some embodiments, the proliferative disease is cancer. Non-limiting examples of cancer include adenocarcinoma, colon cancer, rectal cancer, gastric cancer, lung cancer, renal cell (RC) cancer, liver cancer, kidney cancer, bladder cancer, transitional cell (TC) cancer, prostate cancer, pancreatic cancer, breast cancer, ovarian cancer, thyroid cancer, melanoma, lymphoma, leukemia, and multiple myeloma (MM). The term cancer also refers to cancer cells.

Thus, the invention provides a pharmaceutical composition for use in a method of prophylaxis or treatment of a cancer.

In further embodiments, the disease state or condition is neurofibromatosis. According to the present invention, “neurofibromatosis” (commonly abbreviated NF) refers to a genetically-inherited disorder in which the nerve tissue grows tumors (neurofibromas) that may be benign or may cause serious damage by compressing nerves and other tissues. The disorder affects all neural crest cells (called Schwann cells or melanocytes) and endoneurial fibroblasts. Cellular elements from these cell types proliferate excessively throughout the body, forming tumors. In some embodiments, the disease state or condition is neurofibromatosis type 1, also known as von Recklinghausen disease.

Thus, the invention provides a pharmaceutical composition for use in a method of prophylaxis or treatment of neurofibromatosis.

In another aspect, the invention also contemplates a method for reducing or inhibiting a biological function mediated by LIM Kinase (LIMK), the method comprising administering to a subject (human or non-human) in need thereof an effective amount of a pharmaceutical composition comprising at least one compound of Formula (I) or any other Formulae recited herein.

In some embodiments, the method of the invention is for use in the prophylaxis or treatment of a cancer or neurofibromatosis, as disclosed herein.

In some embodiments, the method further comprises administering to the subject in need thereof an additional anti cancer agent.

The additional anti-cancer agent may be, in accordance with the present invention, any therapeutic agent that can add, additively and/or synergistically, to the usefulness of compound of Formula (I) of the invention in reducing or inhibiting a biological function mediated by LIM Kinase (LIMK).

Some non-limiting examples of anti cancer agents include a cytotoxic agent, a chemotherapeutic agent, an alkylating agent, an antimetabolite, a topoisomerase II inhibitor, a topoisomerase I inhibitor, an antimitotic drug and a platinum derivative.

The composition of the invention or a compound to be administered in accordance with the invention may be administrated by any of the following routes: oral administration, intravenous, intramuscular, intraperitoneal, intratechal or subcutaneous injection; intrarectal administration; intranasal administration; ocular administration or topical administration.

The “therapeutically effective amount” to be administered to said subject (human or non-human) may be determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age and gender, etc.

By yet another aspect, the present invention provides a kit for prophylaxis or treatment of a disease state or condition mediated by LIMK in a patient in need thereof comprising:

a) a therapeutically effective amount of a composition comprising a compound of Formula (I) according to the present invention;

b) instructions for use.

In another aspect of the invention, there is provided a composition, e.g., pharmaceutical composition, comprising at least one compound of Formula (I) and at least one therapeutic agent, as defined hereinabove.

In some embodiments, said therapeutic agent is an anti-proliferative agent, selected to inhibit cancer cell growth. In some embodiments, the anti-proliferative agent is farnesyl thiosalicyclic acid (FTS, Salirasib).

The pharmaceutical compositions comprising a compound of Formula (I) or any other Formulae disclosed herein and at least one therapeutic agent are suitable for use in accordance with the above disclosure.

### BRIEF DESCRIPTION OF THE DRAWINGS

In order to understand the disclosure and to see how it may be carried out in practice, embodiments will now be described, by way of non-limiting example only, with reference to the accompanying drawings, in which:

FIG. 1 presents a scheme depicting Ras-dependent and Ras-independent control pathways of actin dynamics by neurofibromin 1.

FIG. 2 presents a schematic representation showing binding site conservation between EphA3 kinase and its inhibitor, AWL-II-38.3 (compound 2) and the modeled LIMK2. Left drawing represents visualization of the whole binding domain. Right drawing focuses on the AWL-II-38/3EphA3 binding site.

FIG. 3A-B presents an immunoblot and graphical presentation of immunoblots. FIG. 3A demonstrates a typical Western blot, showing protein extracts obtained from cells that were treated with T56-LIMKi (compound 1) or BMS-5 for 2 hours at the indicated concentrations and immunoblotted with specific antibodies (directed against p-cofilin, cofilin or b-tubulin). FIG. 3B shows a quantification of the amount of the detected proteins. Normalization was performed using beta-tubulin. Average inhibition was calculated as a percentage of control (mean±SD, n=3,*P<0.05; **P<0.01 compared to control (Student's t-test)).

FIG. 4 is a graph showing proliferation of NF1−/− MEFs in the presence of T56-LIMKi (compound 1) and FTS. The graph shows the number of NF1−/− MEFs cells, which were grown for 5 days in the absence and in the presence of the indicated concentrations of T56-LIMKi (compound 1) or with 0.1% DMSO (control). Cells were directly counted and typical inhibition curves are shown (means±SEM, n=9; **P<0.01, ***P<0.001).

FIG. 5 is a graph showing statistical analysis of the percentage of cells exhibiting stress fibers. The percentage of cells exhibiting stress fibers (mean±SD, n=3 slides) in a total population of 100 cells was calculated for each slide (*P<0.05, **P<0.01, compared to control (Student's t-test).

FIG. 6 is a graph showing gap width in NF1−/− MEFs cells examined (mean±SD, n=9), expressed as a percentage of the gap at the time of scratching.

FIGS. 7A-B show an image of an anchorage-independent growth assay of NF1−/− MEFs cells in the presence of T56-LIMKi (compound 1) and a graphical representation thereof. FIG. 7A shows images of a typical anchorage-independent growth assay, in which NF1−/− MEFs were grown in soft agar for 14 days in the absence or in the presence of the indicated concentrations of T56-LIMKi (compound 1) (0, 25 and 50 μM), and then stained as described in the Experimental procedures section. FIG. 7B is a graph showing a statistical analysis of the anchorage-independent growth experiment. Columns, mean (n=5); bars, SD; *P<0.001.

FIG. 8A shows western blot levels in of p-cofilin, cofilin, and b-tubulin in Hela cells that were transfected with vehicle-control, LIMK1 or LIMK2. Cells were starved for 24 h and then treated with 50 uM T56-LIMKi (compound 1) for 2 h. FIG. 8B shows quantification of the data depicted in FIG. 9A for p-cofilin only.

FIG. 9 shows that T56-LIMKi inhibits cancer cell growth in vitro. U87-glioblastoma, ST-88-swanoma and Panc-1-pancreatic cancer tumor cell lines were seeded and grown for 5 days in the absence and in the presence of the indicated concentrations of T56-LIMKi (compound 1) or with 0.1% DMSO (control). Cells were directly counted and typical inhibition curves are shown.

FIG. 10 shows that oral administration of T56-LIMKi (compound 1) is not toxic. Nude (CD1-NU) mice were treated with a single dose of oral administration of 20-100 mg/kg of compound 1 in 0.5% carboxymethyl cellulose (CMC) or 0.5% CMC only (control). The mice were weighted and followed for 14 days after the administration. Average weight of treated mice is presented. (n=2).

FIGS. 11A-B show that T-56-LIMKi (compound 1) inhibits proliferation of Panc-1 tumor cells in nude mice. FIG. 11A—Panc-1 cells were implanted s.c. in the right flank of athymic nude mice. After 7 days, the mice were separated randomly into one vehicle-treated control group and two T-56-LIMKi groups (n=8). Daily oral treatment of compound 1 (30 or 60 mg/kg in 0.5% CMC) was given. Tumor volumes (means±S.E.M, * p-value<0.05) during the experiment period are shown. FIG. 11B—The mice were weighted at indicated time point. Presented are average weights of each group (grams±stdev).

### DETAILED DESCRIPTION OF EMBODIMENTS

### Abbreviations

ADF—actin-depolymerizing factor
CSRD—cysteine/serine-rich domain
CTD—C-terminal domain
FCS—fetal calf serum
FTS—S-trans, trans-farnesyl thiosalicyclic acid
GAP—GTPase activating protein
GRD—GAP-related domain
LIMK—LIM kinase

### PDZ

MEF—Mouse embryonic fibroblast
NF1—neurofibromin
NF1^{-/-}—neurofibromin deficient
Pak1—p21-activated kinase 1
PI3K—phospatidylinositol 3-kinase
aa—amino acids
DMEM—Dulbecco's-modified Eagle's medium

The present invention is based on the identification of compounds of Formula (I), which surprisingly were found to have an inhibitory effect on LIM Kinases (LIMK). LIM kinase-1 and LIM kinase-2 belong to a small subfamily of the LIM kinases and have a unique combination of 2 N-terminal LIM motifs and a C-terminal protein kinase domain. LIMK1 and LIMK2 are dual specificity kinases (namely, serine/threonine and tyrosine) that share 70% structural similarity in their kinase domain [5]. Both LIMK1 and LIMK 2 are known as inactivators or inhibitors of the cofilin family of actin-depolymerization factors, by exerting their phosphorylation activity on their substrate, cofilin [9].

Through phosphorylation and inactivation of the actin-depolymerization factor (ADF) cofilin, LIMK1 and LIMK2 are involved, inter alia, in actin cytoskeleton reorganization. LIMK1 also acts to destabilize microtubules and regulates cell motility, including tumor metastasis [2] and plays a regulatory role in tumor cell invasion. It has been shown that the motility of tumor cells correlates with the level of LIMK1 expression and activity [6].

At its active state, cofilin is unphosphorylated, and play a prominent role in promoting actin depolymerization, for example, by inducing severing (depolymerizing) of actin filaments. Cofilin also participates in numerous cellular functions, such as cell migration, cell cycle processes, and neuronal differentiation. At its phosphorylated state, for example by the kinase activity of LIMK1 and/or LIMK2, cofilin is inactive and does not affect the cell cytoskeleton.

### LIM Kinases Activation Pathways

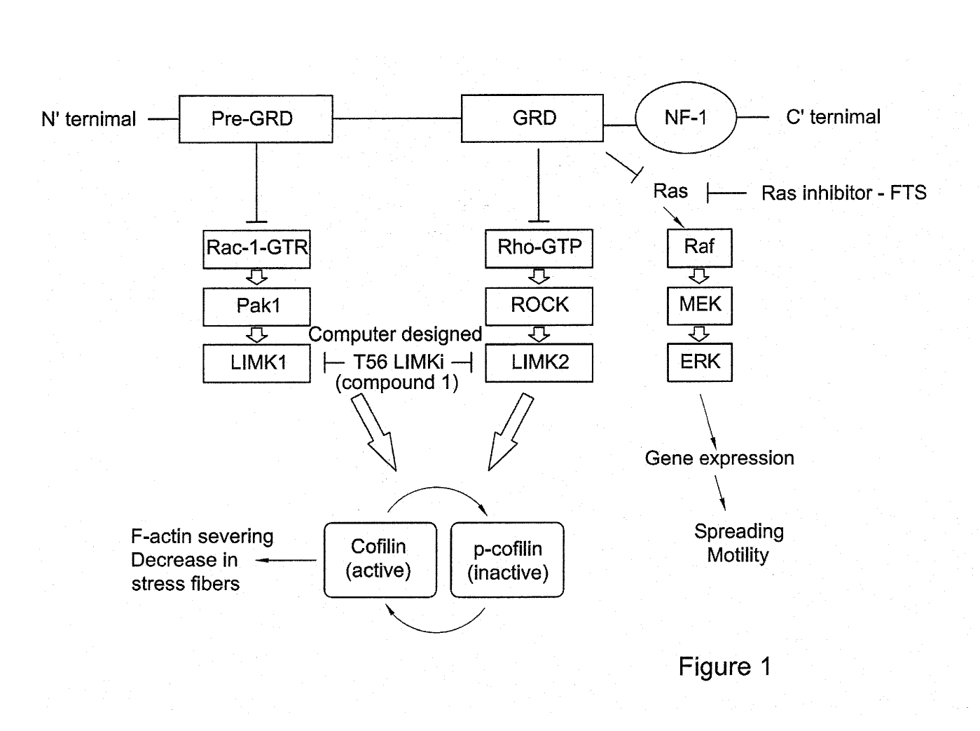

As schematically illustrated in FIG. 1, LIMK2 is activated by the Rho GTPase pathway and LIMK1 is activated by the Rac-1 GTPase pathway. It has been shown that the levels of phosphorylated cofilin (p-cofilin) are high in cells deficient in neurofibromin (NF1−/− cells), suggesting a role for neurofibromin in the LIMK/cofilin pathway. Interestingly, these cells have been shown to present relatively high levels of stress fibers [7].

Neurofibromin 1, the NF1 gene product, is a 2818-amino acid protein [8-10], containing four domains: a cysteine/serine-rich domain (CSRD), a functional Ras GTPase-activating protein (GAP)-related domain (GRD) that follows a pre-GRD domain, a leucine repeat domain, and a C-terminal domain (CTD) (FIG. 1). The GRD domain facilitates GTP hydrolysis by Ras, and exerts the major tumor-suppressor activity through its ability to down-regulate the active Ras proto-oncogene and its pathways. It has been shown that the relatively high levels of active Ras-GTP present in NF1 deficient cells contribute to neurofibromatosis and to cancer in NF1^{-/-} patients [11]. It has also been shown that the high Ras-GTP phenotype of neurofibromin-deficient cells can partially be corrected by the Ras inhibitor S-trans, trans-farnesylthiosalicyclic acid (FTS; Salirasib), and that such treatment leads to the inhibition of Ras downstream effectors. This inhibition leads in turn to a reduced proliferation of NF1^{-/-} cells and tumors.

The present invention provides a composition comprising a compound of Formula (I) for use in reducing or inhibiting a biological function mediated by LIM Kinase (LIMK). In some embodiments, the invention provides a composition comprising a compound as herein described, wherein said reduction results in a restriction, retardation, decrease or diminishing of the biological function mediated by LIM Kinase by at least about 1%-100%, about 5%-95%, about 10%-90%, about 15%-85%, about 20%-80%, about 25%-75%, about 30%-70%, about 35%-65%, about 40%-60% or about 45%-55%. Said restriction, retardation, reduction, decrease or diminishing of a process, a phenomenon or a phenotype mediated by LIM Kinase may also be by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

In some embodiments, the composition according to the invention further comprises an anti-proliferative agent such as Farnesyl Thio Salicyclic acid (FTS; Salirasib). FTS is known to inhibit Ras, which is generally known to be responsible for cell proliferation. As depicted in FIG. 1 and as noted herein above, Neurofibromin appears to regulate cell motility by three distinct GTPase pathways, through two different domains, the GRD and the pre-GRD domains. The first pathway, which is controlled by the GRD domain, is the Ras-Raf-Mek-ERK pathway. This pathway is inhibited by the Ras inhibitor FTS.

Ras regulates the expression of genes that control cell spreading and cell motility [8]. The second pathway is also regulated by the GRD domain, through the Rho-ROCK-LIMK2-cofilin pathway. It has been previously shown that a reduction in p-cofilin levels was not detectable in the presence of the Ras inhibitor FTS [7]. In addition, dominant-negative Ras only partially suppresses the increased p-cofilin levels in NF−/− cells. The third pathway is regulated by the pre-GRD domain and is mediated through Rac-Pak1-LIMK1-cofilin [7].

The inventors have surprisingly shown that a composition comprising a compound of Formula (I) and FTS, provided a synergistic inhibition of NF1-deficient cell proliferation and stress-fiber formation.

LIMK1/2 regulation by NF1 is known to be Ras-independent [7]. Since a synergistic inhibition of NF1-deficient cell proliferation and stress-fiber formation was demonstrated in the presence of a specific compound of Formula (I), namely, compound 1 and the Ras inhibitor FTS, this combination is proposed as a novel approach of potential value for NF1 therapy.

### EXAMPLES

All scientific and technical terms used herein have meanings commonly used in the art unless otherwise specified. The definitions provided herein are to facilitate understanding of certain terms used frequently herein and are not meant to limit the scope of the present disclosure.

Standard molecular biology protocols known in the art not specifically described herein are generally followed essentially as in Sambrook et al., Molecular cloning: A laboratory manual, Cold Spring Harbor Laboratory, New York (1989, 1992), and in Ausubel et al., Current Protocols in Molecular Biology, John Wiley and Sons, Baltimore, Md. (1988).

Standard medicinal chemistry methods known in the art not specifically described herein are generally followed essentially in the series “Comprehensive Medicinal Chemistry” by various authors and editors, published by Pergamon Press.

Standard molecular biology protocols known in the art not specifically described herein are generally followed essentially as in Sambrook & Russell, 2001.

### Experimental Procedures

### Bioinformatics

Homologous proteins were identified by the web-servers “Protein BLAST” and “I-TASSER” [4]. Homology modeling was performed by MODELLER [Eswar, N., et al., Curr. Protoc. Protein Sci. Chapter 2:Unit 2.9 (2007)]. The ZINC database was used to search for a commercially available compound that may be active as inhibitors of LIMK2, as detailed herein below in the Examples section.

### Cell Culture Procedures and Materials

Mouse embryonic fibroblasts (MEFs), both wild-type and NF1 knockout (NF1^{-/-}), were prepared from NF1^{+/-} mice, as described by Shapira et al[14]. Briefly, MEF and HeLa and Panc-1 cells were grown in Dulbecco's-modified Eagle's medium (DMEM), containing 10% fetal calf serum (FCS), 2 mM L-glutamine, 100 units/mL penicillin, and 100 μg/mL streptomycin (DMEM and FCS were both from Biological Industries, Beit Ha Emek, Israel). The cells were incubated at 37° C. in a humidified atmosphere of 95% air and 5% CO₂. The compound T5601640 (defined herein as compound 1 or T56-LIMKi) was purchased from Ambinter (Paris, France). The LIMK inhibitor BMS-5 (Bristol-Myers Squibb) was purchased from SynKinase (Shanghai, China).

### Anchorage-Independent Cell Proliferation in Soft Agar

Noble agar 2% (Difco, Detroit, Mich.) was mixed with DMEM×2 medium, containing 10% FCS, 4 mM L-glutamine, 200 units/mL penicillin, and 0.2 mg/mL streptomycin. The mixture (50 μL) was poured into 96-well plates to provide the agar base at a final agar concentration of 1%. Agar (0.6%) was mixed with DMEM×2, containing cells at a density that provided 8×10⁴ cells per well, and 50 μL of this mixture was seeded on the agar base (at a final concentration of 0.3%). compound 1 mixtures (in DMEM×1 containing 5% FCS) at different compound 1 concentrations were prepared, and 100 μL of each of the mixtures were placed in each well so that the final concentrations of compound 1 were 0, 25 or 50 μM per well. The cells were incubated for 14 days and then stained for 4 hours with 1 mg/mL 344,5-dimethylthiazol-2-yl)-2 5-diphenyltetrazolium bromide (MTT; Sigma-Aldrich, St. Louis, Mo.), which stains active mitochondria in living cells, and the colonies were imaged. Colonies larger than 0.16 mm² (mean±SD, n=5) were counted using Image-Pro Plus software (Media Cybernetics, Carlsbad, Calif.). The average percentage of colonies in each group (means±SD, n=5) was calculated by dividing the number of colonies of a particular treatment and specific group size by the number of colonies of the same size in the corresponding untreated control group.

### Scratch-Induced Migration Assay

Scratch-induced migration assay was performed as described in Goldberg et al. [19]. Briefly, NF1-knockout and wt MEFs were seeded on collagen-covered 35-mm plates at a cell density of 1.5×10⁵ per plate. After 24 hours of incubation, the medium was replaced by FCS (0.5%) containing DMEM, and the cells were treated for 24 hours with compound 1 (50 μM). Three areas were scratched in each plate, creating three gaps of similar widths. The media and the inhibitors were then replenished. Immediately thereafter, and at the time points indicated in the Examples section, phase-contrast images of the plates were obtained with a CCD camera connected to an Olympus fluorescence microscope (×10 objective). The region imaged was marked at time “zero” in order to enable photographing the same area at the different time points, and so that a specific population of migrating cells may be examined. The widths of the gaps treated with the inhibitor were measured at different time points, using the Image-Pro Plus software. The data acquired from the three scratches on each plate were averaged to obtain the mean gap width at a given time. Statistical analysis of the results was performed, of either the mean gap width (in arbitrary units) of compound 1-treated cells relative to the control at different time points (means±SD, n=9) or the percentage of migration, calculated as the width of the gap still open at the final time point, expressed as a percentage of the gap size at zero time for each treatment (means±SD, n=9).

### Western Blot Analysis

NF1−/− MEFs were plated at a density of lx 10⁵ or 5×10⁵ cells in 6-well plates or 10-cm dishes, respectively, and were allowed to grow overnight in a medium containing 10% FCS. The medium was then replaced with a medium containing 0.5% FCS, and the cells were treated for 2 hours with compound 1 at the indicated doses. The cells were then lysed with solubilization buffer (50 mMTris-HCl at a pH of 7.6, 20 mM MgCl₂, 200 mM NaCl, 0.5% NP40, 1 mM Dithiothreitol, and protease inhibitors), and the lysate (50 μg) was subjected to SDS-PAGE and then immunoblotted with one of the following antibodies: anti-p-cofilin (1:1000), anti-cofilin (1:1000), anti-β-tubulin (1:500). The immunoblots were then exposed to peroxidase-goat anti-rabbit IgG (1:2500), and protein bands were visualized by enhanced chemiluminescence and quantified by densitometry (EZ-Qant). Rabbit anti-cofilin and p-cofilin (Ser3) were from Cell Signaling Technolgy (Beverly, Mass.); mouse anti-β-tubulin antibody was from Sigma-Aldrich; peroxidase-goat anti-mouse IgG and peroxidase-goat anti-rabbit IgG were from Jackson ImmunoResearch Laboratories (West Grove, Pa.).

### Fluorescence Staining and Confocal Microscopy

MEFs were seeded on glass coverslips in 6-well plates at the densities of 2.5×10⁴ cells per well. After 24 hours of incubation, the medium was replaced by a medium containing 0.5% FCS and the indicated doses of compound 1. Cells were further incubated for 24 hours and were then fixed, permeabilized, and washed. Rhodamine-labeled phalloidin was added for 30 minutes and the slides were then washed, mounted, and imaged. F-actin was visualized and then photographed under an LSM510 confocal microscope (×63 objective) fitted with rhodamine filters. Statistical analysis was performed by counting 100 cells from each slide, with or without stress fibers, under an Olympus fluorescence microscope. Cells exhibiting stress fibers were expressed as a percentage {mean±SD) of the 100 cells counted (from each slide).

### Animal Studies

Nude CD1-Nu mice (6 weeks old) were housed in barrier facilities on a 12-h light/dark cycle. Food and water were supplied ad libitum. On day zero, 5×10⁶ Panc-1 cells in 0.1 ml of PBS were implanted s.c. just above the right femoral joint When tumor volumes reached values of 0.06-0.07 cm3 (day 0 of compound-1 treatment), the mice were randomly separated into three groups. Control mice received vehicle; compound-1-treated mice received 30 or 60 mg/kg T56-LIMKi (oral administration of 0.1 ml with 0.5% CMC daily). Tumor volume was calculated as (length×width)×[(length+width)÷2].

### Example 1

### Analysis of LIMK1/2 Structures

The inventors have identified a novel inhibitor of LIM domain kinase 2 (LIMK2), by bioinformatic analysis, as described herein below. LIMK2 consists of two LIM domains, a PDZ domain, which is a proline/serine-rich region and a protein kinase domain. The structures of the LIM domains and of the PDZ domains were solved by NMR (PDB ID: 1×6A and 2YUB, respectively). The structure of the protein kinase domain of LIMK2 has yet to be solved.

### Bioinformatic Identification of a LIMK Inhibitor

Solved structures of proteins that are homologous to LIMK were searched in the Protein Data Bank (PDB), while using the Protein BLAST 21] and I-TASSER [5] web-servers. The first homologous structure identified was the recently solved LIMK1 structure (PDB ID: 3S95), which has the best sequence identity with the kinase domain of LIMK2 (64% sequence identity). LIMK1 was crystallized together with the tyrosine kinase inhibitor staurosporine. Staurosporine competes with ATP on binding to the ATP binding sites of many kinases. However, the binding of staurosporine to kinases is characterized by a low selectivity.

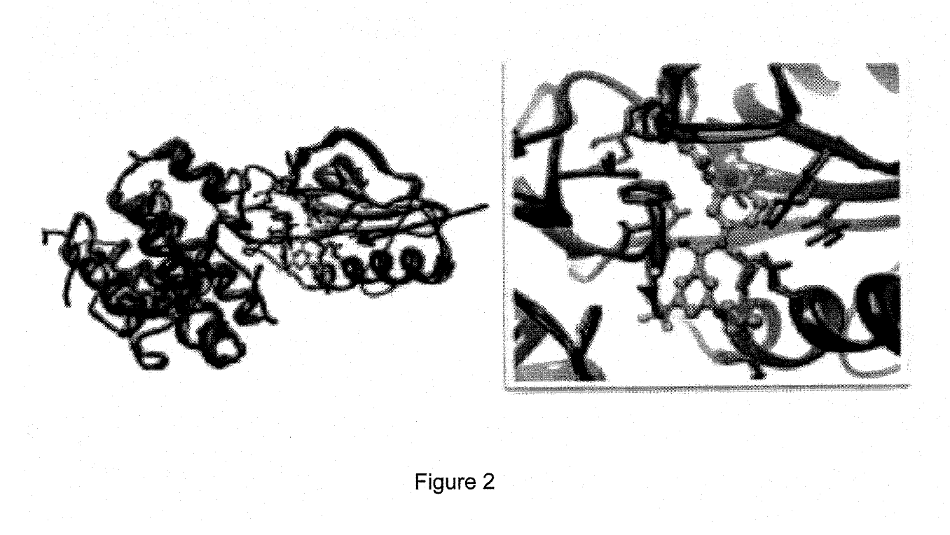

The second LIMK homologue identified was surprisingly found to be the human EphA3 kinase receptor (sharing 31% sequence identity with the kinase domain of LIMK2 (Table 2, below). In one of its PDB-deposited structures, (PDB ID: 3DZQ), EphA3 kinase was crystallized with compound 2 (N-(2-methyl-5-({(3-(4-methyl-1H-imidazol-1-yl)-5-(trifluoromethyl)phenyl)carbonyl}amino)phenyl) isoxazole-5-carbox amide), which is bound in the substrate-binding pocket of EphA3 (FIG. 2, left panel). The MODELLER program [20] was applied to model the structure of the kinase domain of LIMK2 using the EphA3 kinase structure as a template and to compared the inhibitor-binding sites of the two proteins. As demonstrated in Table 2, it was found that the binding site was highly conserved between EphA3 and LIMK2, suggesting that the EphA3 inhibitor may also inhibit LIMK2. Comparison of the binding sites of EphA3 and the protein LIMK1 revealed lower conservation, which may result in a lower affinity of the inhibitor compound 2 for LIMK1 as compared to LIMK2.

TABLE 2
binding site conservation among LIMK1, LIMK2 and EphA3 kinase.
EphA3 residue number

| | 632 | 651 | 652 | 653 | 670 | 673 | 674 | 683 | 697 | 699 |
|---|---|---|---|---|---|---|---|---|---|---|
| EphA3 | F* | A* | I | K* | E#* | I* | M* | I* | I* | T#* |
| LIMK2 | F | V | M | K | E | V | M | L | L | T |
| LIMK1 | G | V | M | K | E | V | M | L | F | T |

EphA3 residue number

| | 700 | 701 | 702 | 737 | 744 | 753 | 762 | 763 | 764 | 765 |
|---|---|---|---|---|---|---|---|---|---|---|
| EphA3 | E | Y | M | L | H | L* | V | S# | D* | F |
| LIMK2 | E | Y | I | L | H | L | V | A | D | F |
| LIMK1 | E | Y | I | L | H | L | V | A | D | F |

Residues that are important for chemical interactions with the inhibitor of EphA3 are marked by:
*hydrophobic interaction;
#hydrogen bond.

### Comparison Between the LIMK2 and EphA3 Binding Sites

A comparison between the inhibitor-binding sites of EphA3 and of the LIMK2 model (FIG. 2) revealed a very high conservation. As demonstrated in Table 2, of the 20 amino acids (aa) in the binding sites, 13 aa were identical (65%), 6 aa had the same hydrophobic property (Ala, Val, Ile and Met), and in only one of the amino acids (namely S135A) the residue differed, resulting in a loss of a hydrogen bond with the inhibitor in LIMK2 (Table 2 and FIG. 2). This high conservation supported our contention that the EphA3 inhibitor, or a similar compound, is likely to inhibit LIMK2. Our model suggested that, unlike other common kinase inhibitors, which compete for the ATP binding site of the protein, the compound identified as a potential LIMK2 inhibitor occupies both the ATP-binding and the substrate-binding sites. This property of the inhibitor might provide enhanced affinity and selectivity toward LIMK2.

As depicted in Table 2, the binding sites of EphA3 and the protein LIMK1 are less well conserved. The aromatic and bulky Phe632 of EphA3 is replaced by Gly346 in LIMK1, and Ile697 of EphA3 is replaced by Phe411 of LIMK1. Without wishing to be bound by theory, these differences might change the shape of the binding site and reduce the affinity of the inhibitor for LIMK1.

The ZINC database was used to search for commercially available compounds that are similar to the EphA3 inhibitor compound 2. Among the compounds that most closely resembled compound 2 was the molecule compound 1. The structures of compound 2 and compound 1 are depicted in Table 1. Upon a careful analysis of the modeled LIMK2 binding site, it appeared that additional compounds may also fit into its active site.

### Example 2

### Compound 1 Reduces Phosphorylated Cofilin (p-Cofilin) in NF1^{-/-} MEFs

The effect of the compound 1 compound on LIMK was then examined by monitoring the phosphorylation level of LIMK's substrate, cofilin. It has been previously shown that the levels of phosphorylated cofilin (p-cofilin) are high in NF1^{-/-} Mouse Embryonic Fibroblasts (MEFs) [7]. Thus, these cells were used herein to examine the impact of compound 1 on phosphorylation of cofilin.

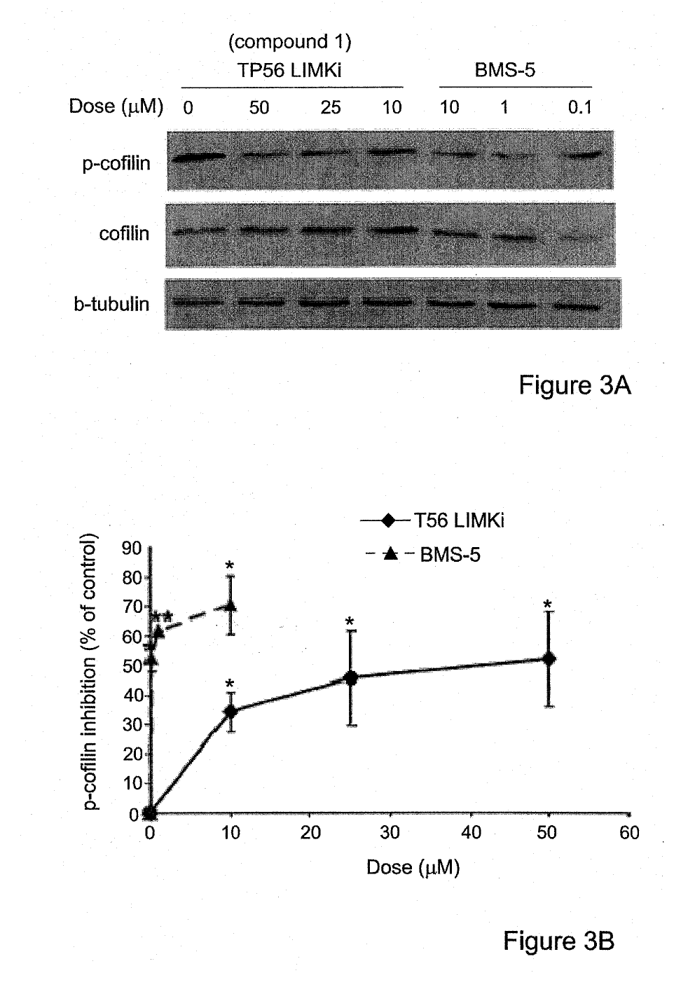

NF1^{-/-} MEFs were serum starved for 24 hours and then incubated for two additional hours in the presence of various concentrations of compound 1 (as described in the “Experimental procedures” section, above). The cells were lysed and subjected to immunoblotting with anti-p-cofilin, anti-cofilin, and anti-β-tubulin (as loading control) antibodies. As shown in FIG. 3, the level of p-cofilin was reduced in the presence of compound 1 (10-50 μM), in a dose-dependent manner. Notably, the compound 1 inhibitor did not affect the amounts of total cofilin (FIG. 3). These results strongly suggested that compound linhibited LIMK, consistently with the predicted model (shown in FIG. 2). A similar experiment performed with the LIMK inhibitor BMS-5 yielded comparable results, except that this inhibitor was more potent than compound 1 (FIG. 3). These findings, taken together, support the predicted LIMK model as well as the predicted LIMK inhibitor. These results did not distinguish, however, between the possible inhibition of LIMK 2, LIMK 1, or both by compound 1.

### Example 3

### Compound 1 Reduces the Number of NF1−/− MEF Cells

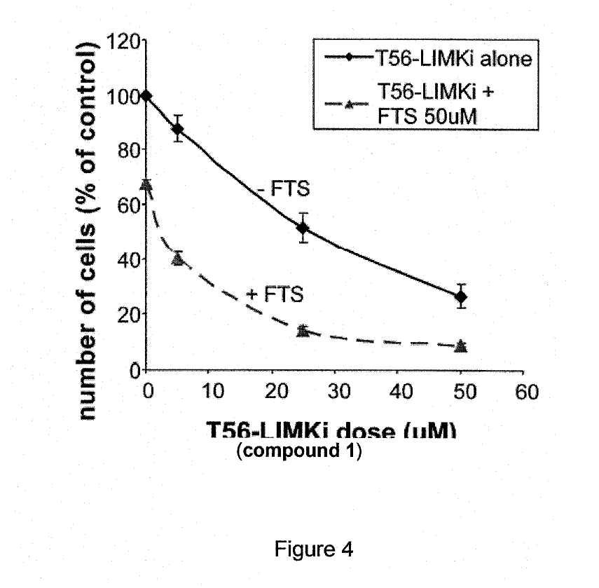

Next, the impact of compound 1 on the growth of NF1^{-/-} MEFs was examined. The cells were plated in 24-well plates at a density of 5×10³ cells per well. As demonstrated in FIG. 4, treatment of the cells with compound 1 at various concentrations resulted in a dose-dependent decrease in cell number, with an IC₅₀ of compound 1 at 30 μM±5.3 (n=9). The effects of the Ras inhibitor S-trans, trans-farnesyl thiosalicyclic acid (FTS) on cell proliferation was also examined, alone and in combination with compound 1. While growth inhibition by compound 1 at 5 μM in the absence of FTS was only 13%±4.9%, it was much higher in its presence (60%±2.5%; FIG. 4). Similar results were obtained for compound 1 at 25 μM (growth inhibition was 51%±2.3% in the absence of FTS and 85.5%±1.1% in its presence; FIG. 4). While FTS alone caused a growth inhibition of only 33%±1.6% (FIG. 4; zero compound 1), the combination of FTS and compound 1 inhibited the growth of the NF^{-/-} cells in a synergistic manner, since, the combination index was lower than 1 (namely 0.82), consistent with the Loewe additively synergistic calculation[15].

### Example 4

### Compound 1 and FTS Induce Synergistic Disassembly of Actin Stress Fibers

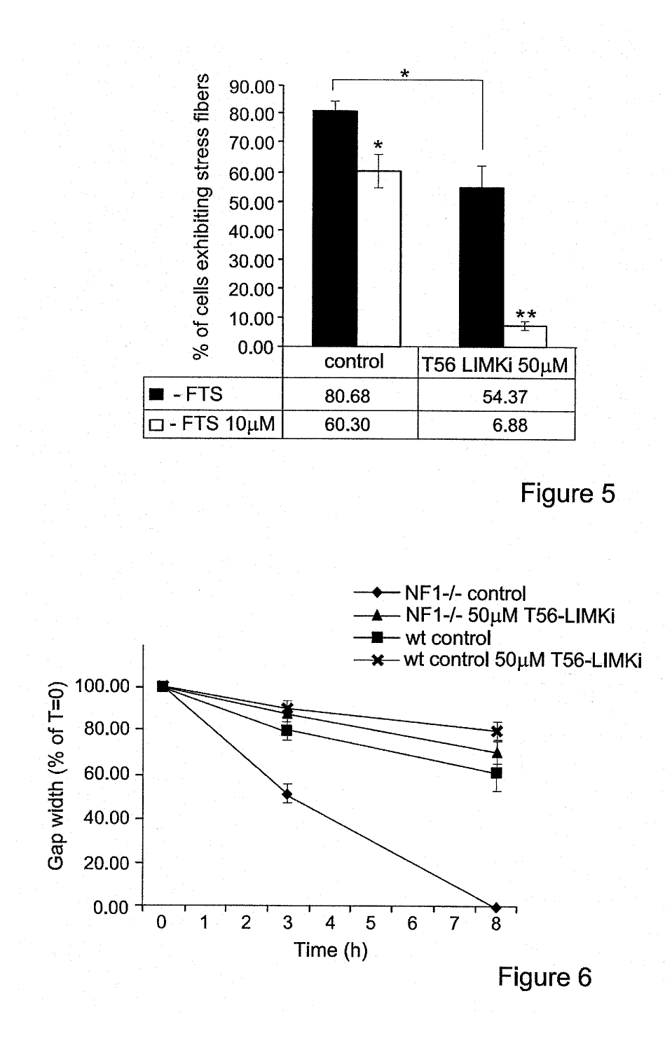

In view of the above results, the effect of compound 1 was also investigated on the actin cytoskeleton, structures that are known to exhibit dramatic changes during cell migration[16]. To this end, control (untreated) NF1^{-/-} MEFs and NF1^{-/-} MEFs, treated with either compound 1, FTS, or their combination were stained with rhodamine-labeled phalloidin, which labels polymeric F-actin. Then, the effect of the inhibitor compound 1 (alone) was examined on the cell cytoskeleton, and specifically on stress-fiber formation. As depicted in FIG. 5, a quantitative analysis of NF^{-/-} MEFs indicated that compound 1 at 50 μM caused a statistically significant reduction in the number of cells exhibiting stress fibers (a decrease of 26%±7.7%; n=300 cells; P<0.05; FIG. 5).

Without wishing to be bound by theory, it is noted that while a decrease of only 30% in the number of cells exhibiting stress fibers was obtained by the relatively high concentration of compound 1 (50 μM, FIG. 5), in the presence of compound 1 at this concentration a 70% reduction in cell proliferation was observed (FIG. 4) as well as about 50% inhibition of cofilin phosphorylation by LIMK (as demonstrated in FIG. 3). These results appear to support the notion that some of the LIMKs were still active. In agreement with previous results, FTS alone decreased stress fiber formation in NF^{-/-} cells by 20%±5.6% (FIG. 5). As demonstrated in FIG. 5, the decrease in stress fiber formation following the combined treatment of compound 1 and FTS was synergistic (74%±1.5%; the combination index calculated by the Loewe additive method was 0.43, i.e., less than 1, indicating synergism). Taken together, the above results support the notion that FTS and LIMK inhibitor operate through different pathways.

### Example 5

### Inhibition of Cell Migration by Compound 1

The effect of the compound 1 inhibitor on cell migration was examined by performing a wound-healing cell migration assay as described by Etienne-Manneville, S. [17], using wild-type (wt) and NF1^{-/-} MEFs. Briefly, the cells were plated in 35-mm plates and incubated with or without the compound 1 inhibitor. After 24 hours, a scratch wound was inflicted on both sets of cells. In order to inhibit cell proliferation, the cells were maintained in medium containing 0.5% FCS, and the width of the gap formed by the scratch was monitored at the indicated time points. FIG. 6 shows the results of a typical experiment using wt and NF1^{-/-} MEFs, with and without compound 1 inhibitor. In the untreated NF1^{-/-} MEF cells the gap closed faster as compared to the treated cells (FIG. 6). For example, in the untreated cells, 50% of the gap was closed within 3 hours, whereas only 10% of the gap was closed in the compound 1-treated cells (FIG. 6). The mobility of the wt MEFs, unlike that of the NF1^{-/-} MEFs, was not affected by the inhibitor (FIG. 6).

### Example 6

### LIMK Inhibition Decreases Anchorage-Independent Cell Growth of NF1^{-/-} MEFs

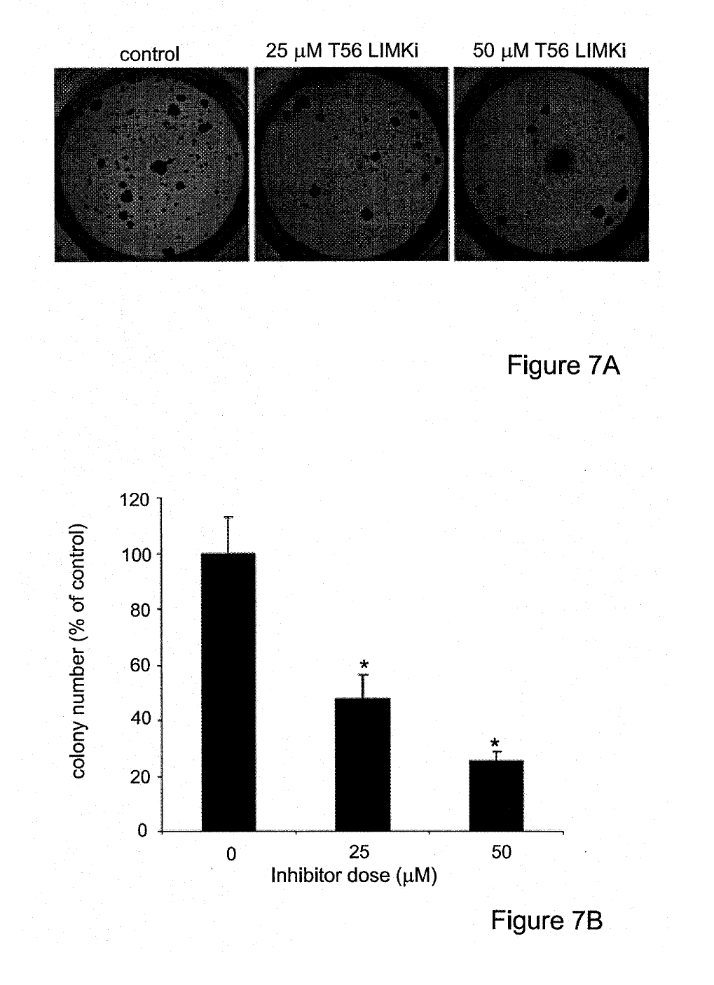

As a measure of cell transformation, the effect of compound 1 on anchorage-independent growth of the NF1^{-/-} MEFs was examined. As demonstrated in FIG. 7, in the absence of compound 1, these cells grew in soft agar and were able to form colonies (it has been previously shown that wt MEFs do not grow in soft agar). However, in the presence of compound 1, colony formation by NF1^{-/-} MEFs was inhibited in a dose-dependent manner (FIG. 7).

### Example 7

### Compound 1 Inhibits LIMK2 and not LIMK1

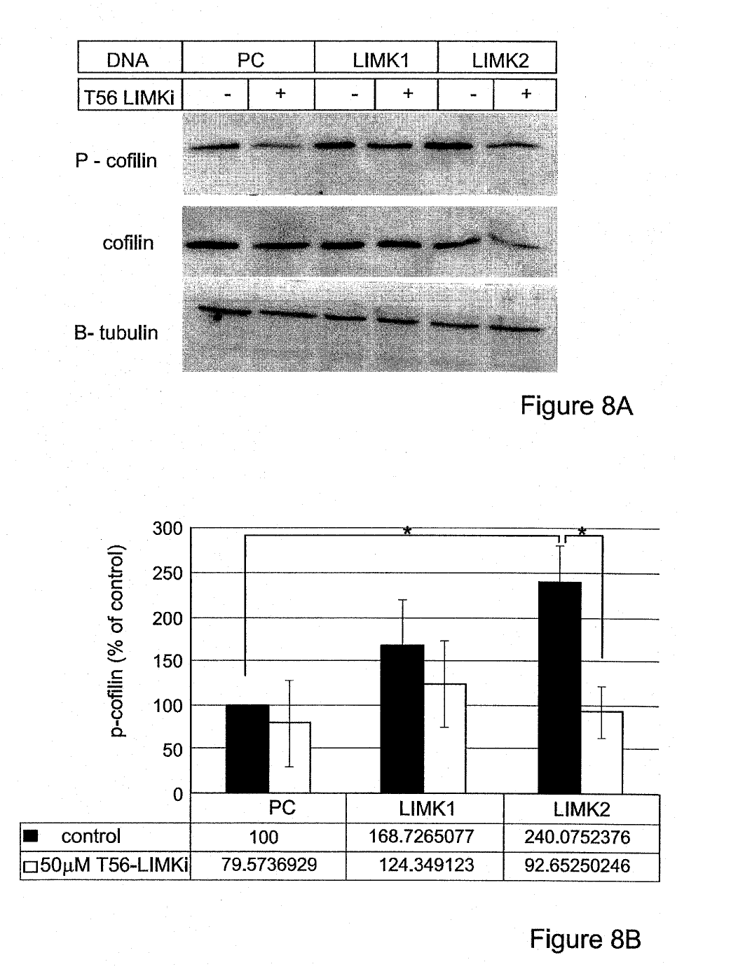

Hela cells were transfected to stably express vehicle-control, LIMK1 or LIMK2. Transfected cells were incubated for 2 h with or without (compound 1). It was found that when the cells expressed compound 1 reduced more efficiently the levels of p-cofilin, compared to LIMK1 and the vehicle (FIG. 8A, B). In fact, p-cofilin levels of the LIMK1 trasfected cells were almost not affected by compound 1.

### Example 8

### Compound 1 Reduces Cell Number a Dose Dependent Manner and without Transfection

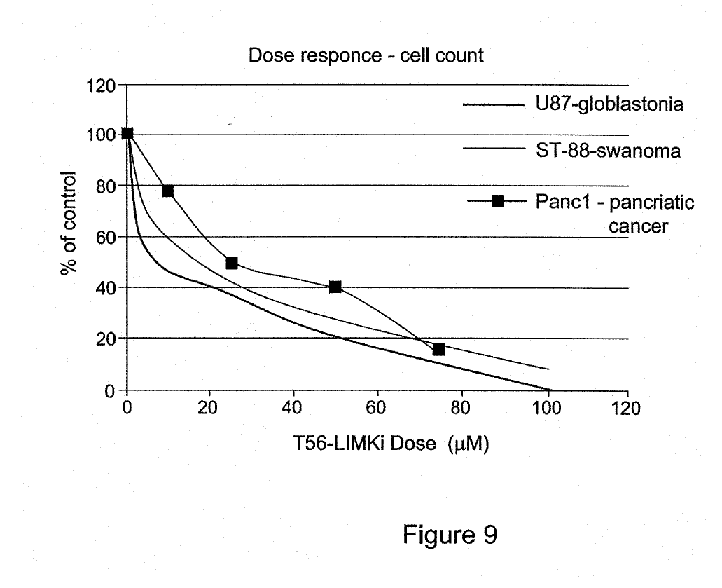

Growth inhibition experiments were performed with compound 1 in U87-glioblastoma, ST-88-swanoma and Panc-1-pancreatic cancer tumor cell lines. These cell lines were chosen because they were found to exhibit relatively low levels of NF1, as in the MEF knockout model. As shown in FIG. 9, compound 1 reduced cell number in all three cell lines in a dose dependent manner, with 1050 of about 25 μM. These results demonstrate the ability of compound 1 to inhibit growth of native tumors without transfection.

### Example 9

### In-Vivo Toxicity Experiments

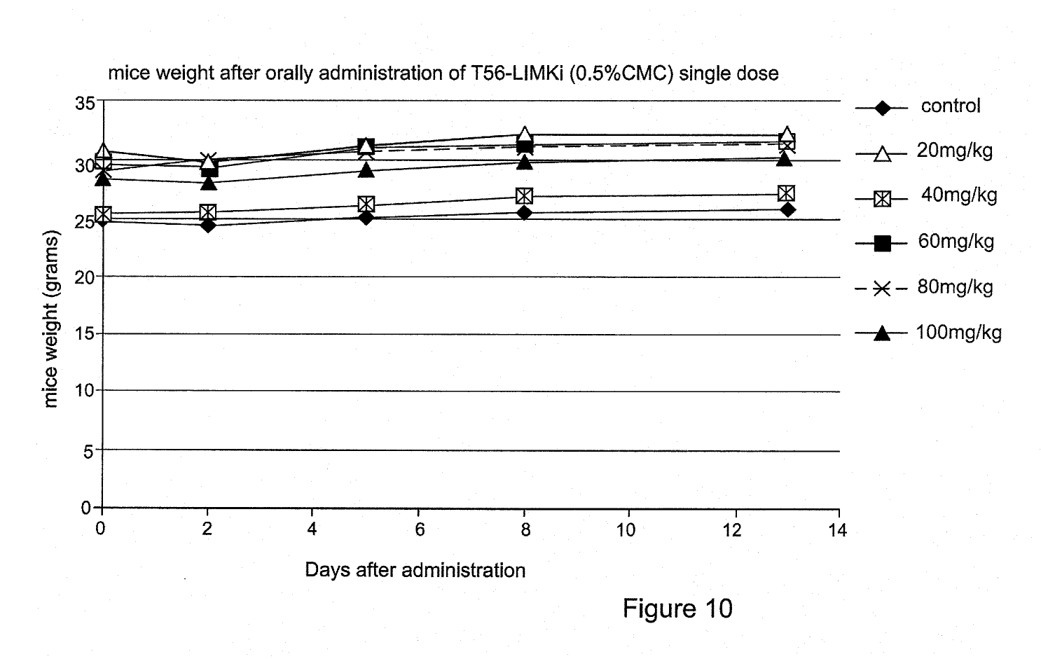

Compound 1 was administrated in 0.5% carboxymethyl cellulose (CMC) (20, 40, 60, 80 or 100 mg/kg), a single dose per mice. The mice were followed for 2 weeks to measure toxic effects. The mice looked normal at all doses, and no weight lost was detected (FIG. 10).

### Example 10

### Orally Administered Compound 1 Inhibits Human Pancreatic Tumor Growth in Nude Mice

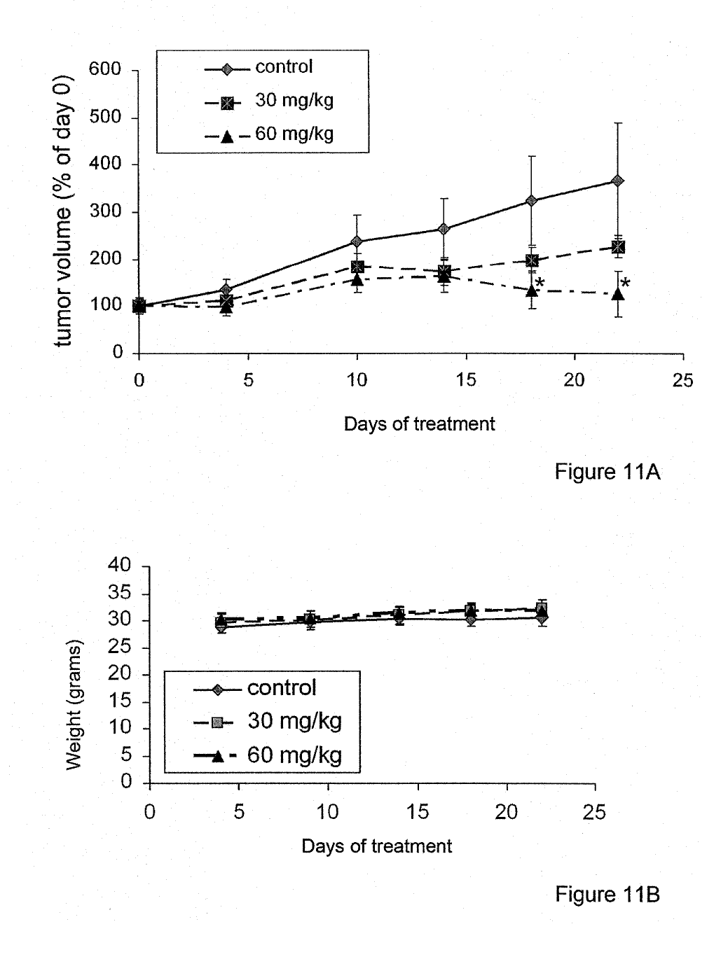

The effects of compound ion human pancreatic tumor growth in nude mice was examined, i.e., on cell transformation in an in vivo model. Mice received 5×10⁶ cells subcutaneously (s.c.) in the right flank. Treatment was started 7 days later, when the mice in the two experimental groups (n=8 per group) received daily oral dose of compound 1 (30 or 60 mg/kg), whereas mice in a control group (n=8) received only vehicle (0.5% carboxymethylcellulose, CMC). As shown (FIG. 11A), at Day 22, tumor growth was inhibited by compound 1, reducing measured tumor size from average size of 745 mm³ in control group, to 488, 268 in 30 and 60 mg/kg, respectively. Compound 1 reduction of the tumor volume of the 60 mg/kg group was significant from day 18. By day 31, 4 out of 8 tumors in the 60 mg/kg treated group disappeared completely. There were no signs of cytotoxicity, and the mouse weight was not affected by compound 1 (FIG. 11B).

1-66. (canceled)

67. A method for reducing or inhibiting a biological function mediated by LIM Kinase (LIMK), the method comprising administering to a subject in need thereof an effective amount of a pharmaceutical composition comprising at least one compound Formula (I), or a pharmaceutically acceptable salt thereof: 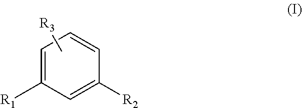
wherein
R₁ and R₂, each independently of the other, is selected from the group consisting of —NHC(═O)R₄ and —C(═O)NHR₅;
R₃ is selected from —H and C₁-C₆alkyl;
R₄ and R₅, each independently of the other, is selected from the group consisting of a substituted or unsubstituted C₆-C₁₂aryl, substituted or unsubstituted C₃-C₅heteroaryl and substituted or unsubstituted C₃-C₅heterocyclic

68. The method according to claim 67, wherein the LIM kinase is LIM kinase-1 (LIMK1) and/or LIM kinase-2 (LIMK2).

69. The method according to claim 67, wherein the biological function to be reduced or inhibited is selected from the group consisting of (a) direct activity of LIMK in phosphorylating actin-depolymerizing factor cofilin, (b) cofilin inactivation, (c) cell motility, (d) actin cytoskeleton reorganization, (e) cell proliferation, (f) cell migration, (g) anchorage-independent cell growth, and (h) tumor progression and metastasis.

70. The method according to claim 67, wherein R₃ represents one, two, three or four substituting groups.

71. The method according to claim 67, wherein each of R₄ and R₅, independently of the other is selected from the group consisting of phenyl, naphthyl, isoxazolyl, and oxazolyl.

72. The method according to claim 71, wherein each of R₄ and R₅, independently of the other, is isoxazolyl, optionally substituted with a C₁₋₆alkyl.

73. The method according to claim 67, wherein R₁ is —NHC(═O)R₄, the compound being a compound of Formula (II): 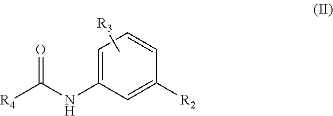
wherein
R₁ is selected from the group consisting of —NHC(═O)R₄ and —C(═O)NHR₅;
R₃ is selected from —H and C₁-C₆alkyl;
R₄ is selected from the group consisting of a substituted or unsubstituted C₆-C₁₂aryl, substituted or unsubstituted C₃-C₅heteroaryl and substituted or unsubstituted C₃-C₅heterocyclic

74. The method according to claim 67, wherein R₁ is —C(═O)NHR₅, the compound being a compound of Formula (III): 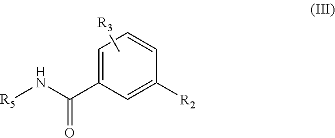
wherein
R₂, is selected from the group consisting of —NHC(═O)R₄ and —C(═O)NHR₅;
R₃ is selected from —H and C₁-C₆alkyl;
R₅, is selected from the group consisting of a substituted or unsubstituted C₆-C₁₂aryl, substituted or unsubstituted C₃-C₅heteroaryl and substituted or unsubstituted C₃-C₅heterocyclic

75. The method according to claim 74, wherein R₂ is selected from the group consisting of —NHC(═O)R₄ and —C(═O)NHR₅.

76. The method according to claim 75, wherein one of said R₅ is a substituted phenyl group, and the other R₅ being selected from the group consisting of phenyl, naphthyl, isoxazolyl, and oxazolyl.

77. The method according to claim 76, wherein one of said R₅ is isoxazolyl optionally substituted with C₁₋₆alkyl, and the other R₅ is —CF₃ substituted phenyl.

78. The method according to claim 73, wherein R₂ is —NHC(═O)R₄, the compound being a compound of Formula (IV): 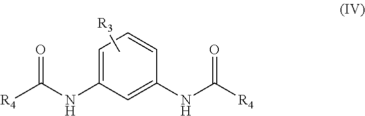
wherein
R₃ is selected from —H and C₁-C₆alkyl;
R₄ each independently of the other, is selected from the group consisting of a substituted or unsubstituted C₆-C₁₂aryl, substituted or unsubstituted C₃-C₅heteroaryl and substituted or unsubstituted C₃-C₅heterocyclic; and wherein each of R₄ may be the same or different.

79. The method according to claim 78, being a compound of Formula (IVa): 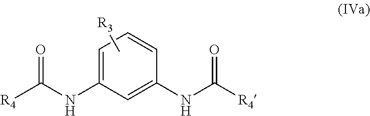
wherein
R₃ is selected from —H and C₁-C₆alkyl;
R₄ is selected from the group consisting of a substituted or unsubstituted C₆-C₁₂aryl, substituted or unsubstituted C₃-C₅heteroaryl and substituted or unsubstituted C₃-C₅heterocyclic; and
R₄¹ is selected from the group consisting of a substituted or unsubstituted C₆-C₁₂aryl, substituted or unsubstituted C₃-C₅heteroaryl and substituted or unsubstituted C₃-C₅heterocyclic.

80. The method according to claim 79, being a compound of Formula (IVb): 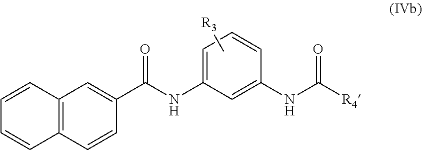
wherein
R₃ is selected from —H and C₁-C₆alkyl;
R₄ each independently of the other, is selected from the group consisting of a substituted or unsubstituted C₆-C₁₂aryl, substituted or unsubstituted C₃-C₅heteroaryl and substituted or unsubstituted C₃-C₅heterocyclic; and wherein each of R₄ may be the same or different.

81. The method according to claim 79, being a compound of Formula (IVc): 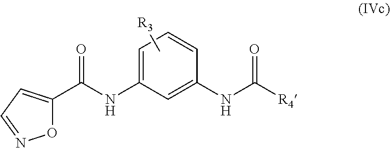
wherein
R₃ is selected from —H and C₁-C₆alkyl;
R₄ each independently of the other, is selected from the group consisting of a substituted or unsubstituted C₆-C₁₂aryl, substituted or unsubstituted C₃-C₅heteroaryl and substituted or unsubstituted C₃-C₅heterocyclic; and wherein each of R₄ may be the same or different.

82. The method according to claim 79, being a compound of Formula (IVd) 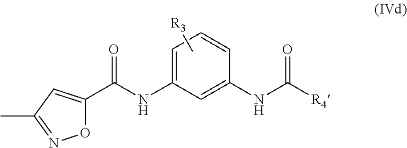
wherein
R₃ is selected from —H and C₁-C₆alkyl;
R₄¹ is selected from the group consisting of a substituted or unsubstituted C₆-C₁₂aryl, substituted or unsubstituted C₃-C₅heteroaryl and substituted or unsubstituted C₃-C₅heterocyclic.

83. The method according to claim 67, being a compound of Formula (V): 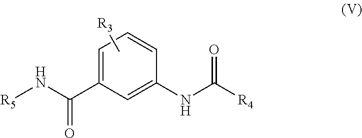
wherein
R₃ is selected from —H and C₁-C₆alkyl;
R₄ and R₅, each independently of the other, is selected from the group consisting of a substituted or unsubstituted C₆-C₁₂aryl, substituted or unsubstituted C₃-C₅heteroaryl and substituted or unsubstituted C₃-C₅heterocyclic

84. The method according to claim 67, wherein the compound is selected the group consisting of compounds herein designated Compound 1, 2, 3, 4, 5, 6, 7, 8, 9, 10, 11, 12, 13, 14, 15, 16 and 17.

85. A pharmaceutical composition comprising at least one compound as defined in claim 67 and at least one additional therapeutic agent.

86. The composition according to claim 85, wherein the therapeutic agent is an anti-proliferative agent.
